(19) United States
(12) Patent Application Publication
(10) Pub. No.: US 2009/0076021 A1
(43) Pub. Date: Mar. 19, 2009

(54) THERAPEUTIC COMBINATIONS AND METHODS FOR CARDIOVASCULAR IMPROVEMENT AND TREATING CARDIOVASCULAR DISEASE

(76) Inventor: Craig F. Plato, Boulder, CO (US)

Correspondence Address: HARNESS, DICKEY, & PIERCE, P.L.C, 7700 Bonhomme, Suite 400, ST. LOUIS, MO, 63105, US

(21) Appl. No.: 12/198,609

(22) Filed: Aug. 26, 2008

Related U.S. Application Data
(63) Continuation of application No. PCT/US2007/005019, filed on Feb. 26, 2007.
(60) Provisional application No. 60/777,387, filed on Feb. 27, 2006.

(51) Int. Cl. A61K 31/197; A61K 31/435; A61K 31/506; A61K 31/166; A61K 31/18; A61K 31/34; A61K 31/427; A61K 31/404; A61K 31/4406; A61K 31/426; A61K 31/41; A61P 9/00

(52) U.S. Cl. 514/252.14; 514/567; 514/296; 514/616; 514/603; 514/427; 514/619; 514/376; 514/415; 514/275; 514/357; 514/371; 514/381

(57) ABSTRACT

A therapeutic combination, useful in a co-therapy method for improving cardiovascular performance and/or treating cardiovascular diseases, is provided comprising a first agent and a second agent, wherein the first agent comprises a histone deacetylase inhibiting agent and the second agent comprises at least one nuclear hormone receptor ligand.

### CROSS-REFERENCE TO RELATED APPLICATIONS

This application is a continuation of PCT International Application PCT/US2007/005019 filed Feb. 26, 2007 which claims priority to U.S. Provisional Patent Application No. 60/777,387 filed Feb. 27, 2006. The entire disclosures of the above applications are incorporated herein by reference.

### FIELD OF THE INVENTION

The present invention generally relates to therapeutic combinations and methods useful for improving cardiovascular performance and treating cardiovascular disease.

### BACKGROUND OF THE INVENTION

Histone deacetylases (“HDACs”) are histone acetyltransferases which transfer an acetyl group to histones thereby playing a role in regulation of gene expression. At present, there are eleven known vertebrate HDACs: HDAC1 through HDAC11. HDAC1, HDAC2, HDAC3, HDAC8, and HDAC11 are class I HDACs. The class I HDACs are ubiquitously expressed, predominantly nuclear, and are believed to function mainly as transcriptional co-repressors. HDAC4, HDAC5, HDAC6, HDAC7, HDAC9, and HDAC10 are class II HDACs. The class II HDACs are tissue specific, suggesting that they may have distinct functions in cellular differentiation and developmental processes. A variety of HDAC inhibitors have been identified. See, for example, Moradeli et al., Histone Deacetylase Inhibitors: Latest Developments, Trends, and Prospects, CURR MED CHEM: ANTI-CANCER AGENTS 5(5):529-560 (2005).

Nuclear hormone receptors are ligand-activated transcription factors that regulate gene expression by interacting with specific DNA sequences upstream of their target genes. As early as 1968 a two-step mechanism of action was proposed for these receptors based upon the observation of an inactive and an active state of the receptors. The first step involves activation through binding of the hormone; the second step consists of receptor binding to DNA and regulation of transcription.

By interacting with their nuclear receptors, hormones regulate a wide variety of physiological functions including metabolism, growth, and cell differentiation. Because disruptions in these functions often cause disease, the study of nuclear hormone receptors and the hormones which bind them are a rapidly developing area of research.

Thyroid cells produce the thyroid hormones, thyroxine (“T4”) and triiodothyronine (“T3”). Thyroid hormones exert effects on the heart and the cardiovascular system. T3 has been shown to act on the cardiac myocyte via genomic (nuclear) and nongenomic pathways. T3 binds to nuclear thyroid hormone receptors (“TRs”) which in turn bind to thyroid hormone response elements in the promoter region of thyroid hormone-responsive genes. In the presence of T3, TRs activate transcription by recruiting coactivator complexes, and in the absence of T3, TRs repress transcription by recruiting corepressor complexes.

The prevalence of cardiovascular diseases, conditions, and adverse effects is increasing in the patient population. Therefore, there is a need for drug therapies useful for improving cardiovascular performance, and treating cardiovascular disease.

U.S. Pat. No. 6,544,957 identifies the compound 6-(1,3-Dioxo-1H,3H-benzo[de]isoquinolin-2-yl)-hexanoic acid hydroxyamide, termed “scriptaid” as a histone deacetylase inhibitor, and then mentions a composition containing scriptaid and an expression construct encoding a therapeutic polypeptide to increase production of a polypeptide. However, the patent discloses neither a combination of an HDAC inhibitor and thyroid hormone, nor a use of any combination of agents treating cardiovascular diseases.

Thyroid hormone (TH) is known to be involved in histone modification. More specifically, Alan P. Wolffe, Nature (News and Views) Vol. 287, 16-17 (1997), mentions that the presence of TH helps recruitment of histone acetyltransferases to relieve transcriptional repression. However, this publication does not teach a combination comprising a HDAC inhibitor and a thyroid hormone to treat cardiovascular disease.

Mitchell A. Lazar, J. Clin. Invest. 112:497-499 (2003) mentions that TH induces binding of transcriptional coactivators possessing histone acetyltransferase activity. However, this publication does not teach a combination comprising a HDAC inhibitor and a thyroid hormone to treat cardiovascular disease.

### SUMMARY OF THE INVENTION

There is now provided, a therapeutic combination comprising a first agent and a second agent, wherein the first agent comprises a histone deacetylase inhibiting agent and the second agent comprises at least one nuclear hormone receptor ligand, and the second agent is present in a sub-optimal dose.

There is further provided, a co-therapy method for improving cardiovascular performance, comprising administering to an animal a first amount of a first agent comprising a histone deacetylase inhibiting agent and a second amount of a second agent comprising at least one nuclear hormone receptor ligand, wherein the first amount and second amount together provide a therapeutically effective combination of the first agent and second agent.

There is still further provided, a co-therapy method for treating cardiovascular disease, comprising administering to an animal a first amount of a first agent comprising a histone deacetylase inhibiting agent and a second amount of a second agent comprising at least one nuclear hormone receptor ligand, wherein the first amount and second amount together provide a therapeutically effective combination of the first agent and second agent.

### BRIEF DESCRIPTION OF THE DRAWINGS

FIG. 1 is a graphical representation of the results obtained from the study described in the Example herein.

FIG. 2 is a graphical representation of the results obtained from the study described in the Example herein.

### DETAILED DESCRIPTION OF THE INVENTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In various aspects of the invention, a therapeutic combination and method is provided for improving cardiovascular performance, and preventing and/or treating cardiovascular disease.

The methods of this invention are particularly suitable for use with humans, but may be used with other animals, particularly mammals, such as, for example, non-human primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

Cardiovascular performance may be improved in a number of ways. For example, cardiovascular performance is improved by preventing and/or alleviating any cardiovascular-associated condition or symptom. Preventing in this context means reducing the risk of, delaying the onset of, and/or keeping a subject from developing the cardiovascular disease state, condition, or symptom thereof.

Cardiovascular performance may also be improved by enhancing the cardiovascular fitness of healthy subjects. Examples of cardiovascular improvement include, but are not limited to, increasing a maximum rate of oxygen consumption (VO₂ₘₐₓ), increasing partial pressure of oxygen (PO₂), and increasing exercise time.

Improvement of cardiovascular performance further includes the reduction or elimination of risks or adverse events associated with any cardiovascular treatment or regime.

Further exemplary improvements of cardiovascular performance include the reduction or alleviation of one or more of the following cardiovascular-associated conditions, symptoms, or adverse events: decreased exercise capacity, severe recurrent headache, decreased blood ejection volume, increased left ventricular end diastolic pressure, increased pulmonary capillary wedge pressure, decreased cardiac output, low cardiac index, increased pulmonary artery pressures, increased left ventricular end systolic and diastolic dimensions, increased left and right ventricular wall stress, increased wall tension, decreased quality of life, disease-related morbidity and mortality, confusion and fatigue, chest pain, dypsnea, irregular heartbeat, and blood in the urine.

Improvement of cardiovascular performance can be measured in variety of ways known to those skilled in the art. Exemplary methods to measure improvement of cardiovascular performance include, but are not limited to, echocardiogram, electrocardiogram, 6-minute walk test, cardiac index, cardiac output, LVEDP (left ventricular end diastolic pressure), ejection fraction, PAP (pulmonary arterial pressure), and echo based measurements including cardiac dimension, ventricular filling velocity via Doppler (mitral velocity), decreased dypsnea and pulmonary edema.

Further, the present invention can be used to treat or alleviate cardiovascular disease states. Treating includes ameliorating and/or eradicating the cardiovascular disease state, condition, or symptom thereof.

Exemplary cardiovascular disease states or conditions which may be improved include, but are not limited to diastolic heart failure, diastolic dysfunction, cardiac fibrosis, hypertrophy, impaired ventricular relaxation, impaired ventricular filling, pulmonary hypertension, pulmonary edema, shortness of breath, hypertension of all etiologies, acute coronary syndrome (including unstable angina and non-Q wave infarction), myocardial infarction, heart failure, systolic heart failure, stroke, occlusive stroke, hemorrhagic stroke and combinations thereof.

The term “therapeutic combination” refers to a plurality of agents that, when administered to a subject together or separately, are co-active in bringing therapeutic benefit to the subject. Such administration is referred to as “combination therapy,” “co-therapy,” “adjunctive therapy” or “add-on therapy.” For example, one agent can potentiate or enhance the therapeutic effect of another (i.e. provide a synergistic effect), or reduce an adverse side effect of another, or one or more agents can be effectively administered at a lower dose than when used alone, or can provide greater therapeutic benefit than when used alone, or can complementarily address different aspects, symptoms or etiological factors of a disease or condition.

As such, in one embodiment a therapeutic combination is provided comprising a first agent and a second agent, wherein the first agent comprises a HDAC inhibiting agent and the second agent comprises at least one nuclear hormone receptor ligand.

In another embodiment, a co-therapy method for improving cardiovascular performance is provided comprising administering to a subject a first amount of a first agent comprising a HDAC inhibiting agent and a second amount of a second agent comprising at least one nuclear hormone receptor ligand, wherein the first amount and second amount together provide a therapeutically effective combination of the first agent and second agent.

In yet another embodiment, a co-therapy method for treating cardiovascular diseases is provided comprising administering to a subject a first amount of a first agent comprising a HDAC inhibiting agent and a second amount of a second agent comprising at least one nuclear hormone receptor ligand, wherein the first amount and second amount together provide a therapeutically effective combination of the first agent and second agent.

The co-therapy method can have one or more of a number of objectives and results, including without limitation to increase the efficacy, decrease the side effects, or enhance the onset of action of the first agent or the second agent, for example.

As discussed above, the first agent comprises a HDAC inhibiting agent, wherein the HDAC inhibiting agent comprises an HDAC inhibitor (i.e., the HDAC inhibiting agent comprises one or more independently selected HDAC inhibitors). An HDAC inhibitor can be an HDAC inhibiting compound or a derivative thereof (e.g., a salt, solvate, hydrate, or prodrug of the HDAC inhibiting compound). Depending on the particular HDAC inhibiting compound, a salt of the compound may be advantageous due to one or more of the salt's physical properties, for example, enhanced pharmaceutical stability in differing temperatures and humidities, or a desirable solubility in water or oil. Preferably the salt of the HDAC inhibiting compound is a pharmaceutically-acceptable salt.

In some embodiments, the HDAC inhibiting agent inhibits HDAC 1. In some embodiments, the HDAC inhibiting agent inhibits HDAC 2. In some embodiments, the HDAC inhibiting agent inhibits HDAC 3. In some embodiments, the HDAC inhibiting agent inhibits HDAC 4. In some embodiments, the HDAC inhibiting agent inhibits HDAC 5. In some embodiments, the HDAC inhibiting agent inhibits HDAC 6. In some embodiments, the HDAC inhibiting agent inhibits HDAC 7. In some embodiments, the HDAC inhibiting agent inhibits HDAC 8. In some embodiments, the HDAC inhibiting agent inhibits HDAC 9. In some embodiments, the HDAC inhibiting agent inhibits HDAC 10. In some embodiments, the HDAC inhibiting agent inhibits HDAC 11.

In some embodiments, the HDAC inhibiting agent inhibits two or more of HDAC 1, HDAC 2, HDAC 3, HDAC 4, HDAC 5, HDAC 6, HDAC 7, HDAC 8, HDAC 9, HDAC 10, and HDAC 11. In some such embodiments, the HDAC inhibiting agent inhibits two or more class I HDACs (e.g., HDAC 1, HDAC 2, HDAC 3, HDAC 8, or HDAC 11). In other such embodiments, the HDAC inhibiting agent inhibits two or more class II HDACs (e.g., HDAC 4, HDAC 5, HDAC 7, HDAC 9, or HDAC 10). In further such embodiments, the HDAC inhibiting agent inhibits one or more class I HDACs and one or more class II HDACs.

In some embodiments, the HDAC inhibiting agent comprises a class I HDAC inhibitor (i.e., the HDAC inhibiting agent comprises one or more independently selected class I HDAC inhibitors). A class I HDAC inhibitor is an inhibitor that inhibits one or more class I HDACs (e.g., HDAC 1, HDAC 2, HDAC 3, HDAC 8, or HDAC 11).

In some embodiments, the HDAC inhibiting agent comprises a class II HDAC inhibitor (i.e., the HDAC inhibiting agent comprises one or more independently selected class II HDAC inhibitors). A class II HDAC inhibitor is an inhibitor that inhibits one or more class II HDACs (e.g., HDAC 4, HDAC 5, HDAC 7, HDAC 9, or HDAC 10).

In some embodiments, the HDAC inhibiting agent comprises one or more HDAC inhibitors independently selected from the group consisting of class I HDAC inhibitors and class II HDAC inhibitors (e.g., the HDAC inhibiting agent comprises one class I HDAC inhibitor, or the HDAC inhibiting agent comprises two class II HDAC inhibitors, or the HDAC inhibiting agent comprises one or more class I HDAC inhibitors and one or more class II HDAC inhibitors).

In some embodiments, the HDAC inhibiting agent comprises one or more HDAC inhibitors independently selected from the group consisting of HDAC 1 inhibitors, HDAC 2 inhibitors, HDAC 3 inhibitors, HDAC 4 inhibitors, HDAC 5 inhibitors, HDAC 6 inhibitors, HDAC 7 inhibitors, HDAC 8 inhibitors, HDAC 9 inhibitors, HDAC 10 inhibitors, and HDAC 11 inhibitors.

In some embodiments, the HDAC inhibiting agent comprises an HDAC 1 inhibitor (i.e., the HDAC inhibiting agent comprises one or more HDAC 1 inhibitors). In some embodiments, the HDAC inhibiting agent comprises an HDAC 2 inhibitor. In some embodiments, the HDAC inhibiting agent comprises an HDAC 3 inhibitor. In some embodiments, the HDAC inhibiting agent comprises an HDAC 4 inhibitor. In some embodiments, the HDAC inhibiting agent comprises an HDAC 5 inhibitor. In some embodiments, the HDAC inhibiting agent comprises an HDAC 6 inhibitor. In some embodiments, the HDAC inhibiting agent comprises an HDAC 7 inhibitor. In some embodiments, the HDAC inhibiting agent comprises an HDAC 8 inhibitor. In some embodiments, the HDAC inhibiting agent comprises an HDAC 9 inhibitor. In some embodiments, the HDAC inhibiting agent comprises an HDAC 10 inhibitor. In some embodiments, the HDAC inhibiting agent comprises an HDAC 11 inhibitor.

In some embodiments of the methods of prevention and treatment, the HDAC inhibiting agent comprises a hydroxamic acid HDAC inhibitor (i.e., the HDAC inhibiting agent comprises one or more hydroxamic acid HDAC inhibitors and, optionally, one or more additional HDAC inhibitors). Hydroxamic acid HDAC inhibitors suitable for the use with the invention include, for example:

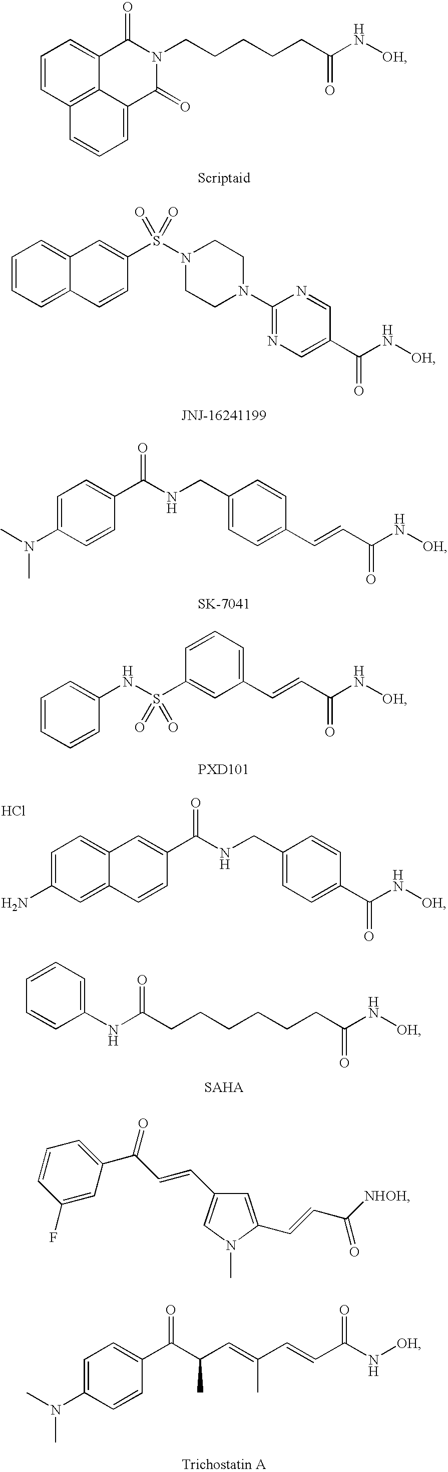

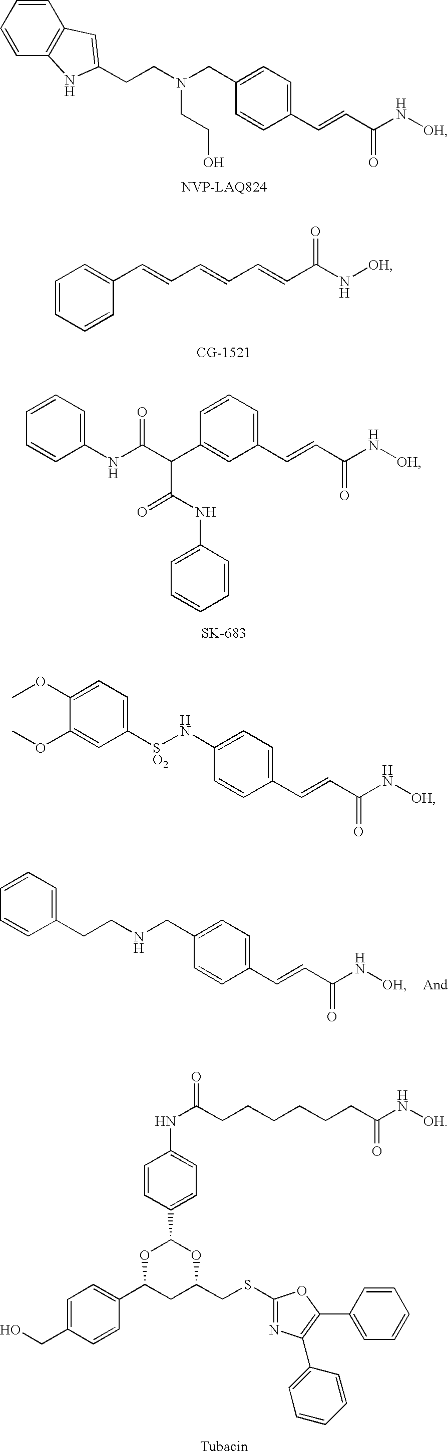

In some embodiments, the HDAC inhibiting agent comprises an aniline amide HDAC inhibitor (i.e., the HDAC inhibiting agent comprises one or more aniline amide HDAC inhibitors and, optionally, one or more additional HDAC inhibitors). Aniline amide HDAC inhibitors suitable for the methods of prevention and treatment of this invention include, for example:

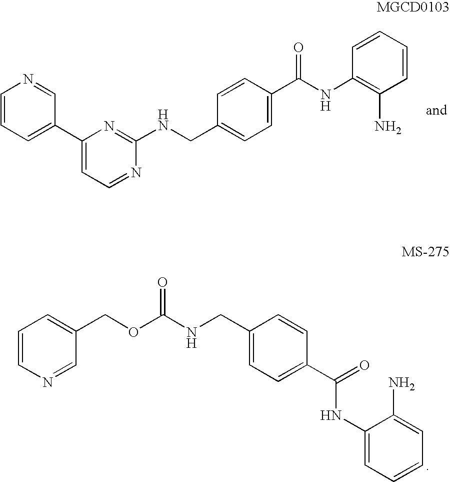

In some embodiments, the HDAC inhibiting agent comprises a ketone HDAC inhibitor (i.e., the HDAC inhibiting agent comprises one or more ketone HDAC inhibitors and, optionally, one or more additional HDAC inhibitors). Ketone HDAC inhibitors suitable for the methods of prevention and treatment of this invention include, for example:

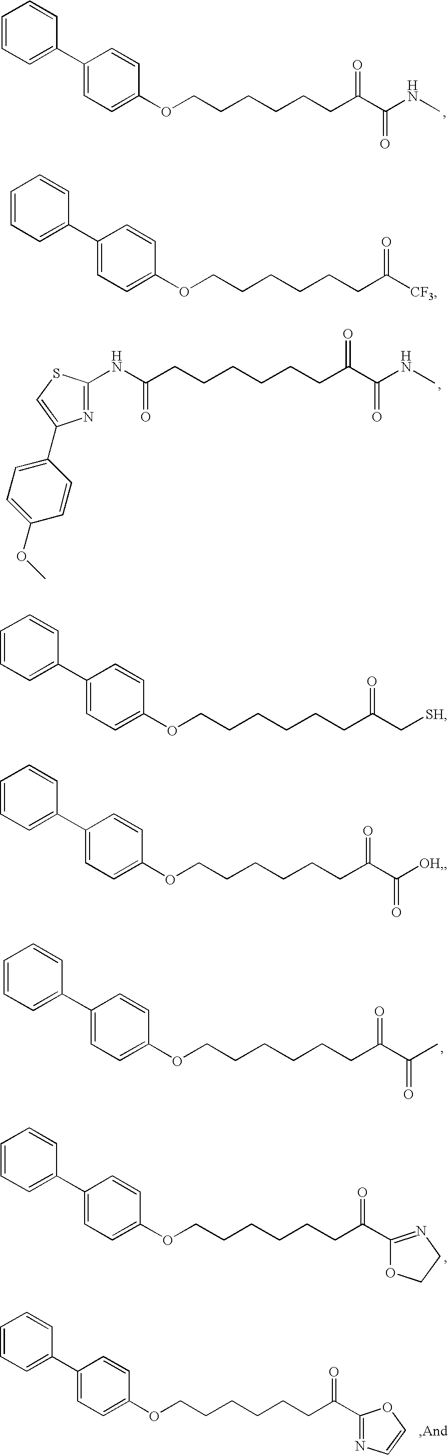

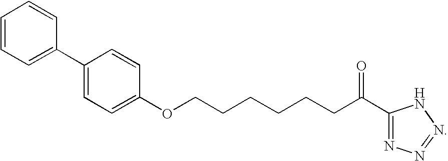

In some embodiments, the HDAC inhibiting agent comprises a fatty acid HDAC inhibitor (i.e., the HDAC inhibiting agent comprises one or more fatty acid HDAC inhibitors and, optionally, one or more additional HDAC inhibitors). Fatty acid HDAC inhibitors suitable for the methods of prevention and treatment of this invention include, for example:

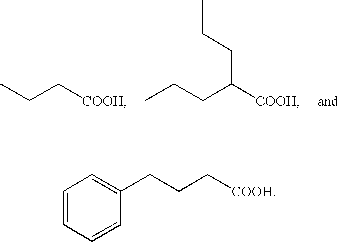

In some embodiments, the HDAC inhibiting agent comprises one or more HDAC inhibitors independently selected from the group consisting of hydroxamic acid HDAC inhibitors, aniline amide HDAC inhibitors, ketone HDAC inhibitors, and fatty acid inhibitors.

In some embodiments, the HDAC inhibiting agent comprises one or more inhibitors discussed in Moradeli et al., Histone Deacetylase Inhibitors: Latest Developments, Trends, and Prospects, CURR MED CHEM: ANTI-CANCER AGENTS 5(5):529-560 (2005).

As discussed above, the second agent comprises at least one nuclear hormone receptor ligand. Exemplary nuclear hormone receptor ligands include thyroid hormone, DITPA, GC-1, vitamin D, all-trans-retinoic acid, 9-cis-retinoic acid, and including small molecule hormone mimetics. In a particular embodiment, the second agent comprises at least one thyroid hormone. And in yet another embodiment, the second agent comprises T3.

In some embodiments, the first agent inhibits one or more of HDAC 1, HDAC 2, HDAC 3, HDAC 4, HDAC 5, HDAC 6, HDAC 7, HDAC 8, HDAC 9, HDAC 10, and HDAC 11, and the second agent comprises a thyroid hormone.

In some embodiments, the first agent inhibits one or more of HDAC 1, HDAC 2, HDAC 3, HDAC 4, HDAC 5, HDAC 6, HDAC 7, HDAC 8, HDAC 9, HDAC 10, and HDAC 11, and the second agent comprises T3.

It should be understood that the second agent of this invention may be used with any combination of the first agent as previously described herein.

In some embodiments, the second agent is present in a sub-optimal dose, where sub-optimal dose implies a dose of the second agent insufficient to produce modulate cardiac performance, chamber size or pressures.

In some embodiments, the first agent is administered at a dose of about 0.01 to about 100 mg/day and the second agent is administered at a single dose of about 0.1 to about 100 μg/day.

In another embodiment, the combination of the present invention results in the modification of α-myosin heavy chain (MHC) transcription. In particular, the combination results in an increase in both α-MHC transcription and protein expression.

In some embodiments, a therapeutic agent used in the combinations and methods of this invention is administered as part of a pharmaceutical composition (or medicament) that further comprises one or more pharmaceutically-acceptable carriers, diluents, wetting or suspending agents, vehicles, and/or adjuvants (the carriers, diluents, wetting or suspending agents, vehicles, and adjuvants are sometimes being collectively referred to in this patent application as “carrier materials”); and/or other active ingredients.

Co-administration of a first and a second agent, as in the co-therapy method of the invention, comprises administration of the agents in amounts sufficient to achieve or maintain therapeutically effective concentrations, e.g., plasma concentrations, in the subject in need thereof. Co-administration can comprise one or both of simultaneous and subsequent (i.e., sequential) administration. Simultaneous administration can comprise administration of the agents as a single composition or as different compositions (see below) “at the same time” within a treatment period. Sequential administration can comprise administration of the agents at different times, for example “at intervals” within a treatment period.

Administration “at the same time” includes administration of the first and second agents literally “at the same time,” but also includes administration directly one after the other, in any order. Administration “at intervals” includes administration of the first agent and the second agent at different times, separated for example by an interval of about 1 h, about 6 h, about 12 h, about 1 day, or about 1 month at the maximum.

The first agent and the second agent may be formulated in one pharmaceutical preparation (single dose form) for administration at the same time or may be formulated in two distinct preparations (separate dose forms) for administration at the same time or sequentially.

The two distinct preparations in the separate dose forms may be administered by the same route or by different routes. The first and second agents of the present invention may be administered orally, but the invention is not limited to any route of administration, so long as the route selected results in effective delivery of the drug so that the stated benefits are obtainable. Thus administration of the combination can illustratively be parenteral (e.g., intravenous, intraperitoneal, subcutaneous or intradermal), transdermal, transmucosal (e.g., buccal, sublingual or intranasal), intraocular, intrapulmonary (e.g., by inhalation), rectal, or any combination thereof. If the combination is delivered orally, any suitable orally deliverable dosage form can be used, including without limitation tablets, capsules (solid- or liquid-filled), powders, granules, syrups and other liquids, etc.

Separate dose forms can optionally be co-packaged, for example in a single container or in a plurality of containers within a single outer package, or co-presented in separate packaging (“common presentation”). As an example of co-packaging or common presentation, a kit is contemplated comprising, in separate containers, the first agent and the second agent. In another example, the first agent and the second agent are separately packaged and available for sale independently of one another, but are co-marketed or co-promoted for use according to the invention. The separate dose forms may also be presented to a subject separately and independently, for use according to the invention.

Depending on the dosage forms, which may be identical or different, e.g., fast release dosage forms, controlled release dosage forms or depot forms, the first agent and the second agent may be administered on the same or on different schedules, for example on a daily, weekly or monthly basis. Therefore, the administration interval in a co-therapy method of the invention may depend on the administration schedules or on the dosage forms.

### EXAMPLE

The following example is merely illustrative, and not limiting to the remainder of this disclosure in any way.

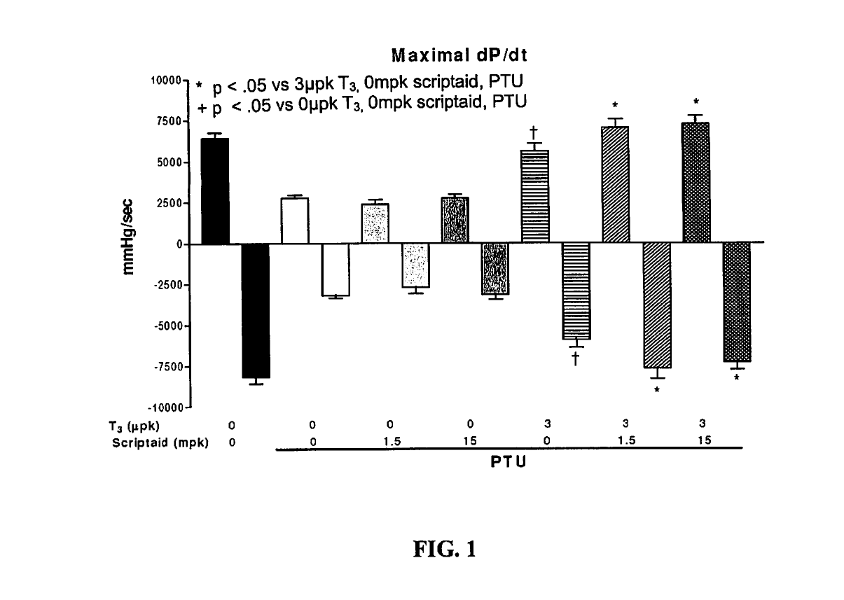

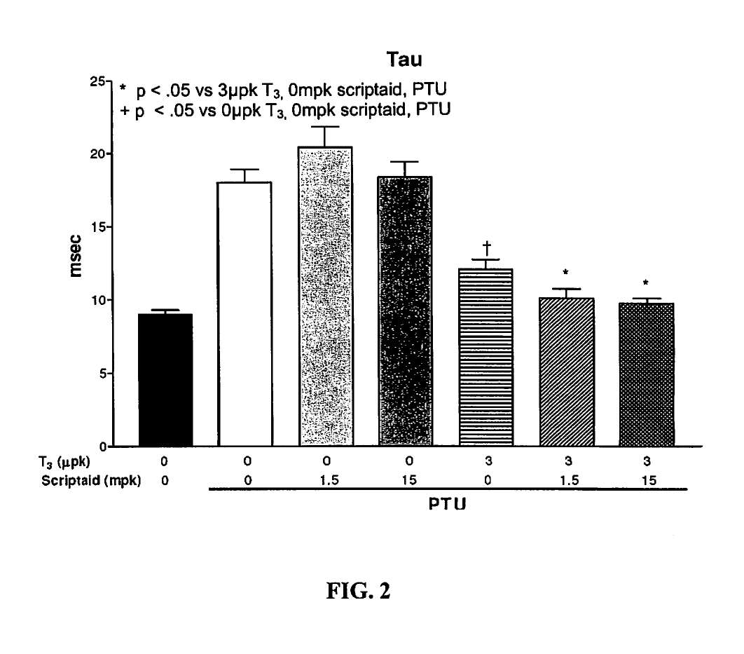

Male S.D. rats (7 wks old) were rendered hypothyroid by being fed PTU diet (n 48; iodine deficient, 0.15% propylthiouracil) or normal chow (n=8) for 2 weeks. PTU fed rats were then separated into weight-matched groups the day prior to inception of the study, and treated for 4 days with vehicle (0.05 mL/100 g BW i.p.; 20% Cremophor EL; 20% ethanol; 60% H₂O) or T₃ (3 μg/kg) and scriptaid (1.5, 15 mg/kg) or its vehicle (100% DMSO; 0.04 mL/100 g BW i.p.). Because of differential pharmacodynamics of T₃ and scriptaid, each animal received T₃ or its vehicle at least 3 hours prior to receiving scriptaid or its vehicle. Following 4 days of treatment, core temperature, systemic hemodynamics, and cardiac performance data were collected under isoflurane anesthesia using the Millar direct catheter system no less two hours following scriptaid administration. At the conclusion of the experiment the animals were sacrificed, and blood and tissues collected for morphological and biochemical analysis. Hypothyroid rats have impaired systolic and diastolic cardiac performance relative to euthyroid control rats, while scriptaid alone exerted no effects on either systolic or diastolic cardiac performance. Exogenous T3 increased both maximal positive and negative dP/dt (See FIG. 1); indices of systolic and diastolic performance, respectively while tau (an index of myocardial relaxation) was also improved (See FIG. 2). Coadministration of scriptaid significantly enhanced the effects of T3 treatment on each index (maximal +/−dP/dt, tau) of cardiac performance, indicating that low doses of HDAC inhibitors potentiate the effects of nuclear hormone receptor ligands.

1-72. (canceled)

73. A therapeutic combination comprising a first agent and a second agent, wherein the first agent comprises a histone deacetylase inhibiting agent and the second agent comprises at least one nuclear hormone receptor ligand, and the second agent is present in a sub-optimal dose.

74. The combination of claim 73, wherein the second agent comprises at least one thyroid hormone.

75. The combination of claim 74, wherein the at least one thyroid hormone is tri-iodothyronine.

76. The combination of claim 73, wherein the first agent comprises one or more histone deacetylase inhibitors independently selected from the group consisting of histone deacetylase 1 inhibitors, histone deacetylase 2 inhibitors, histone deacetylase 3 inhibitors, histone deacetylase 4 inhibitors, histone deacetylase 5 inhibitors, histone deacetylase 6 inhibitors, histone deacetylase 7 inhibitors, histone deacetylase 8 inhibitors, histone deacetylase 9 inhibitors, histone deacetylase 10 inhibitors, and histone deacetylase 11 inhibitors.

77. The combination of claim 73, wherein the first agent comprises one or more histone deacetylase inhibitors independently selected from the group consisting of:

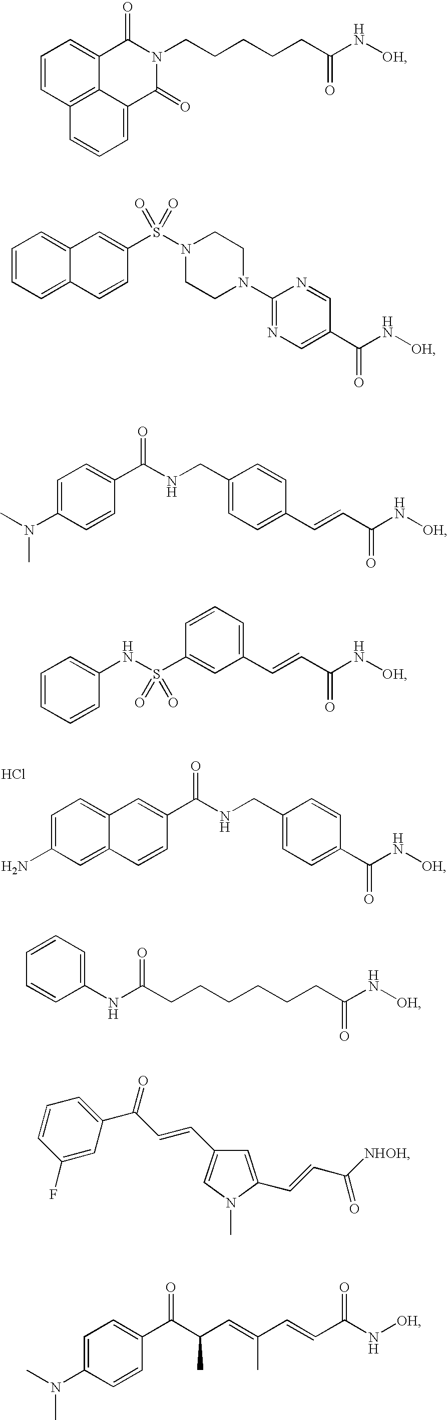

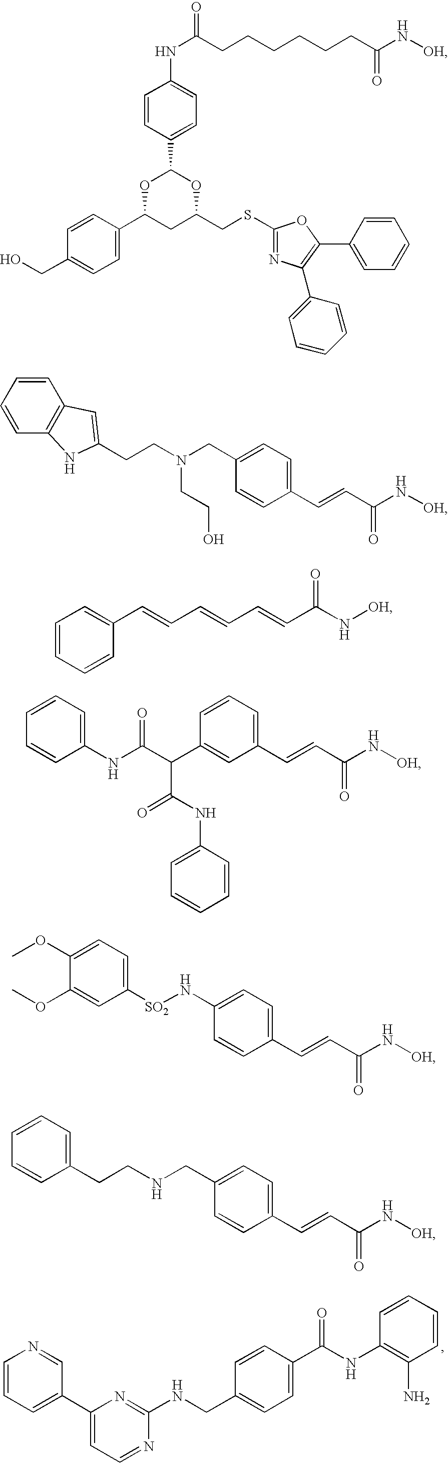

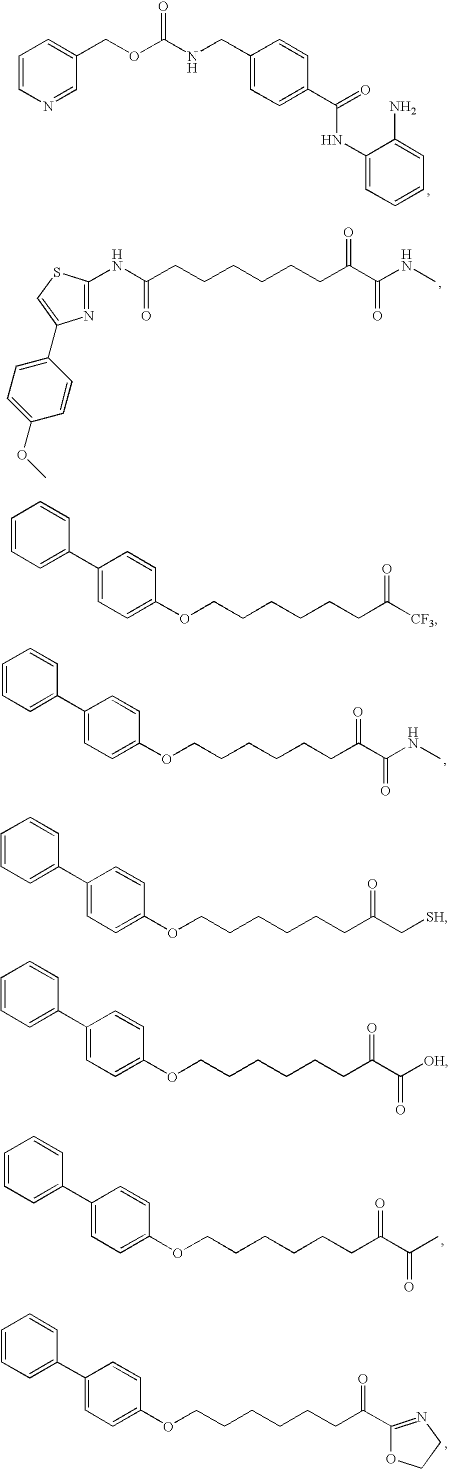

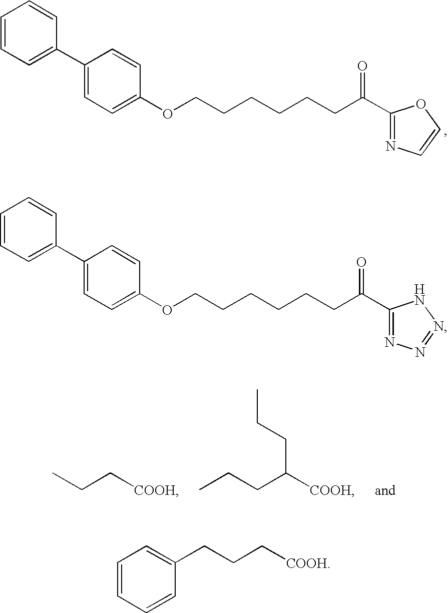

78. The combination of claim 77, wherein the second agent comprises tri-iodothyronine.

79. A co-therapy method for improving cardiovascular performance or treating cardiovascular disease, comprising administering to a human a first amount of a first agent comprising a histone deacetylase inhibiting agent and a second amount of a second agent comprising at least one nuclear hormone receptor ligand, wherein the first amount and second amount together provide a therapeutically effective combination of the first agent and second agent.

80. The method of claim 79, wherein the second agent comprises at least one thyroid hormone, and the amount of the second agent is a sub-optimal dose.

81. The method of claim 80, wherein the at least one thyroid hormone is tri-iodothyronine.

82. The method of claim 79, wherein the first agent comprises one or more histone deacetylase inhibitors independently selected from the group consisting of histone deacetylase 1 inhibitors, histone deacetylase 2 inhibitors, histone deacetylase 3 inhibitors, histone deacetylase 4 inhibitors, histone deacetylase 5 inhibitors, histone deacetylase 6 inhibitors, histone deacetylase 7 inhibitors, histone deacetylase 8 inhibitors, histone deacetylase 9 inhibitors, histone deacetylase 10 inhibitors, and histone deacetylase 11 inhibitors.

83. The method of claim 79, wherein the first agent comprises one or more histone deacetylase inhibitors independently selected from the group consisting of:

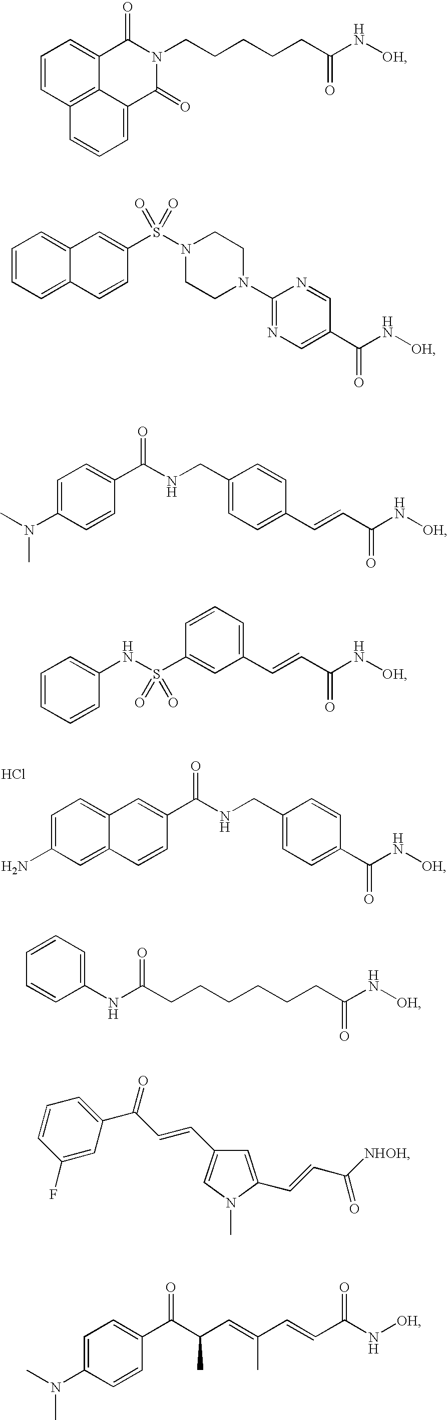

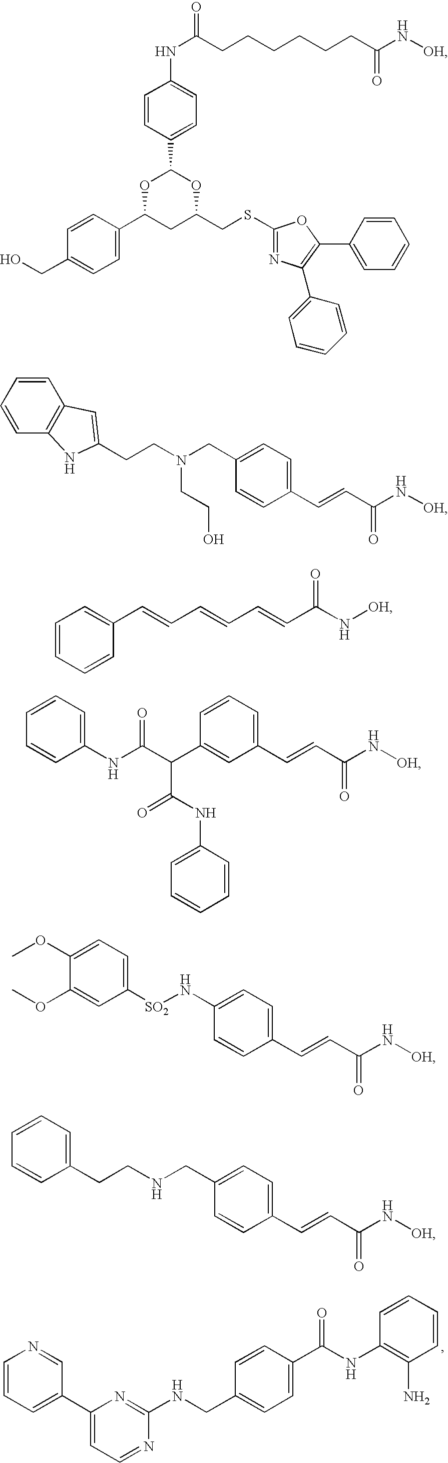

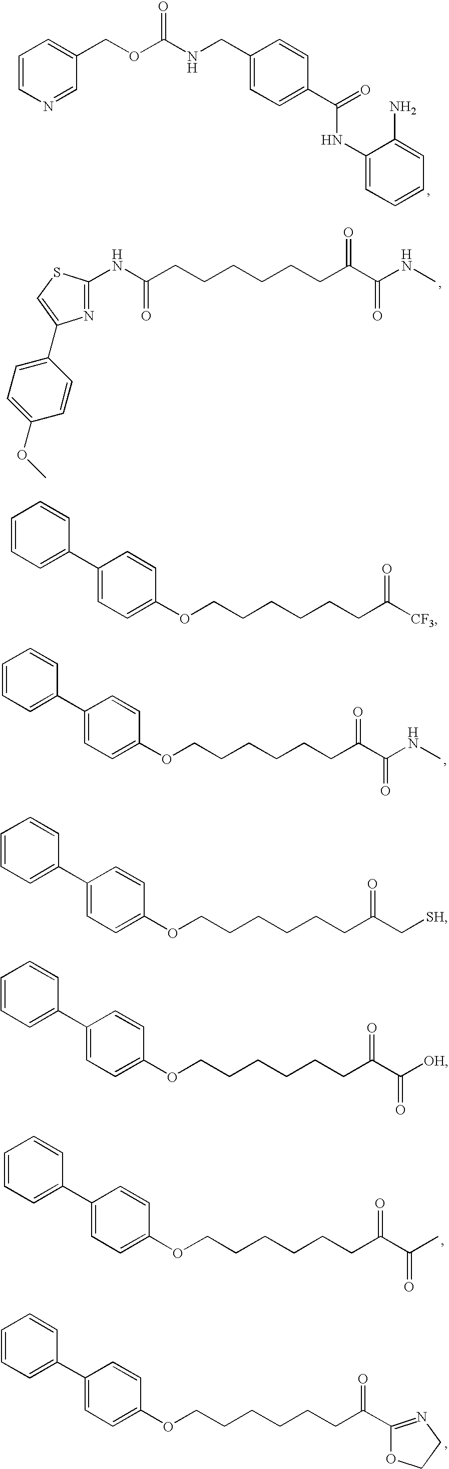

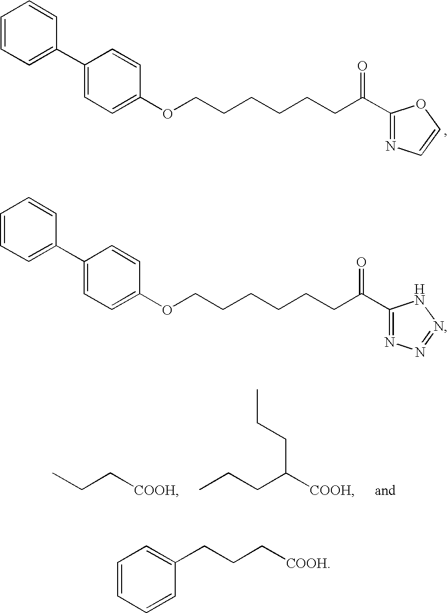

84. The method of claim 83, wherein the second agent comprises tri-iodothyronine and the amount of the second agent is a sub-optimal dose.

85. The method of claim 79, wherein the cardiovascular disease treated is selected from the group consisting of diastolic heart failure, diastolic dysfunction, cardiac fibrosis, hypertrophy, impaired ventricular relaxation, impaired ventricular filling, pulmonary hypertension, pulmonary edema, shortness of breath, hypertension of all etiologies, acute coronary syndrome including unstable angina and non-Q wave infarction, myocardial infarction, heart failure, systolic heart failure, stroke, occlusive stroke, and hemorrhagic stroke.

86. The method of claim 79, wherein the administration of the first agent and the second agent results in modification of α-myosin heavy chain (MHC) transcription.

87. The method of claim 79, wherein the first agent is administered at a dose of about 0.01 to about 100 mg/day and the second agent is administered at a single dose of about 0.1 to about 100 μg/day.
